(19) United States
(12) Patent Application Publication
(10) Pub. No.: US 2025/0115567 A1
(43) Pub. Date: Apr. 10, 2025

(54) PROCESS FOR PREPARING POLYCYCLIC ODORANTS

(71) Applicant: SYMRISE AG, Holzminden (DE)

(72) Inventors: Bernd HÖLSCHER, Halle (DE); Tobias WAGNER, Hellental (DE)

(21) Appl. No.: 18/281,162

(22) PCT Filed: Apr. 3, 2021
(86) PCT No.: PCT/EP2021/058820

§ 371 Date: Sep. 8, 2023

(51) Int. Cl. C07D 307/79

(52) U.S. Cl. CPC C07D 307/79

(57) ABSTRACT

A process is proposed for preparing compounds of formula (I), (I) where the respective radicals in the compound of formula (I) are as follows: R1, R2, R3 and R4 each independently denote hydrogen, methyl, ethyl, propyl, isopropyl, butyl, sec-butyl, isobutyl, acetyl, formyl or cyano, comprising or consisting of the following steps: (A) etherification of a phenol derivative to obtain an allyl ether derivative; (B) Claisen rearrangement and subsequent cyclization of the ether from step (A); (C) acetylation or formylation of the intermediate from step (B), with steps (A) and (B) being effected in a one-pot reaction.

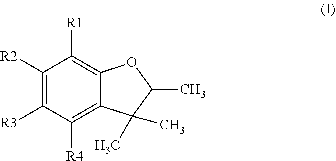

### FIELD OF THE INVENTION

The invention relates to the preparation of compounds of the formula (I) which are particularly suitable for use as fragrances.

### BACKGROUND OF THE INVENTION

Despite a large number of existing fragrances and aromas, there is still a general need for further fragrances and aromas in the perfume industry. In particular, there is thus a need for fragrances with musky scent notes, in particular those fragrances that are able to produce other interesting odoriferous notes in addition to a musky scent note (in fragrance mixtures) and to expand the possibilities for the perfumer with their novel or original scent properties. In particular, there is interest in fragrances with musky scent notes which are able to form a harmonious combination with woody and/or flowery fragrances and/or other musky fragrances. The different olfactory aspects and notes should preferably be superimposed in order to thereby produce an overall complex olfactory impression.

EP 2 641 903 A1 describes the compounds of the formula (I). However, the preparation thereof has to date required a very complex method, which includes, inter alia, a 3-stage synthesis. This method is not only extremely time-consuming but also only affords a moderate yield.

Consequently, the object of the present invention was to provide a method which overcomes the disadvantages of the prior art. In particular, the object of the present invention was to provide a method which enables the compounds of the formula (I) to be prepared simply and more rapidly and at the same time maximizes the yield of compounds of the formula (I).

### DESCRIPTION OF THE INVENTION

This object is fully achieved by the claims of the present invention. The present invention relates to a method for preparing compounds of the formula (I)

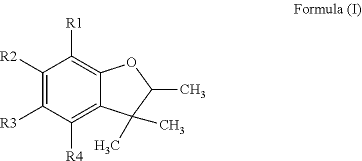

wherein for the radicals of the compound of the formula (I), it applies in each case that R1, R2, R3 and R4 are each independently hydrogen, methyl, ethyl, propyl, isopropyl, butyl, sec-butyl, tert-butyl, isobutyl, acetyl, formyl or cyano, comprising or consisting of the following steps
(A) etherification of a phenol derivative to obtain an allyl ether derivative;
(B) Claisen rearrangement and subsequent cyclization of the ether from step (A);
(C) acetylation or formylation of the intermediate from step (B),
wherein the steps (A) and (B) are carried out in a one-pot reaction.

The inventors have surprisingly discovered that the method of the present invention is not only simpler, more rapid and more cost-efficient compared to the methods of the prior art to date, but at the same time also increases the yield of the compounds of formula (I) to over 70%. This applies in particular to the methods described in EP 2 641 903 A1. The increase in yield was particularly surprising in that steps (A) and (B), i.e. the etherification and subsequent Claisen rearrangement and cyclization, are carried out in a one-pot reaction and consequently the intermediate obtained is not isolated.

In the context of the present invention, a one-pot reaction is a chemical synthesis which is characterized in that as far as possible all the required reagents and solvents are mixed in a vessel at the start and then allowed to react (usually with stirring/mixing and heating or cooling). Sometimes individual components are only added in the course of the operation (preferably via a dropping funnel or other metering device). Consequently, one-pot reactions do not require isolation of intermediates, which can save material, time, and energy. In other words, steps (A) and (B) of the present method are carried out without isolation of intermediates obtained in step (A).

The compounds of the formula (I) may optionally be present in each case as pure stereoisomers or as a mixture of stereoisomers. In particular, a compound of the formula (I) may exist as an enantiomer or a mixture of enantiomers.

Step (C) is preferably an acetylation of the intermediate from step (B).

In a preferred embodiment of the invention, it applies for the compound of the formula (I) or one, more than one or all compounds of the formula (I), in each case mutually independently that:
R1 is methyl, ethyl, propyl, isopropyl, butyl, sec-butyl, tert-butyl or isobutyl, preferably methyl, ethyl, isopropyl or tert-butyl and/or
R2 is hydrogen, methyl, formyl or acetyl, preferably hydrogen, formyl or acetyl, and/or
R3 is hydrogen, methyl, formyl or acetyl, preferably hydrogen, formyl or acetyl, and/or
R4 is hydrogen, methyl, formyl or acetyl, preferably hydrogen, formyl or acetyl.

In a further preferred embodiment of the invention, the compound of the formula (I) or one, more than one or all compounds of the formula (I) is selected or each independently selected from the group consisting of the following compounds 1 to 3:

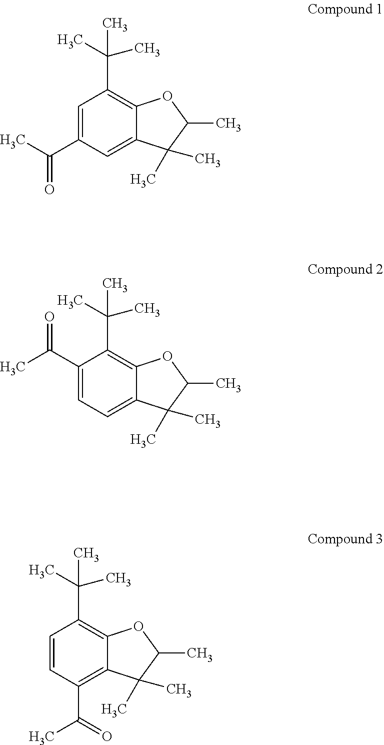

An exemplary sequence of the preparation method according to the invention is shown below in FIG. 1:

FIG. 1: Example of the sequence of the preparation method according to the invention

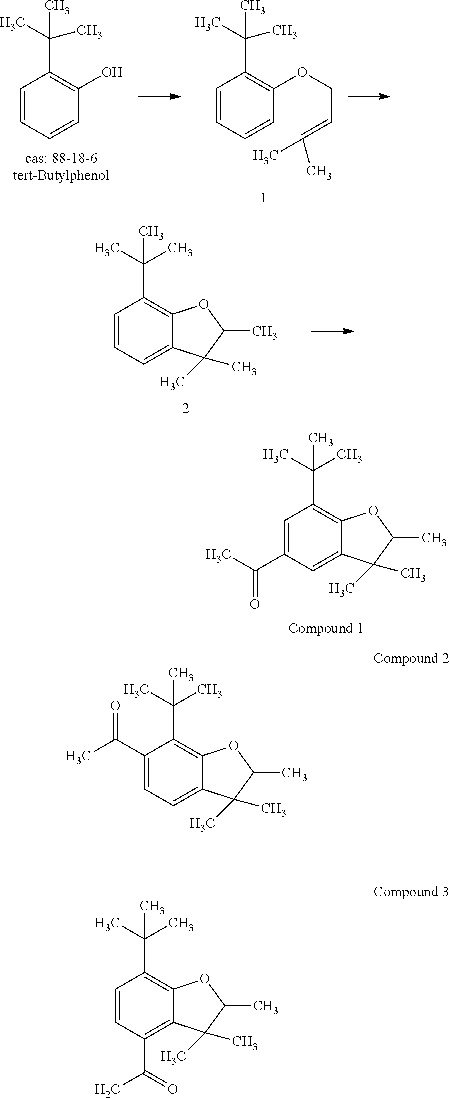 [SMILES: CC(C)(C)c1ccccc1O ; CC(C)=CCOc1ccccc1C(C)(C)C ; CC1Oc2c(C(C)(C)C)cccc2C1(C)C ; CC(=O)c1cc(C(C)(C)C)c2c(c1)C(C)(C)C(C)O2 ; CC(=O)c1ccc2c(c1C(C)(C)C)OC(C)C2(C)C ; CC(=O)c1ccc(C(C)(C)C)c2c1C(C)(C)C(C)O2]

As can be seen in FIG. 1, the phenol derivative is first converted to the corresponding allyl ether derivatives (step 1). This is followed by the Claisen rearrangement with subsequent cyclization (step 2). The dihydrobenzofurans thus obtained can then be acetylated or formylated under standard conditions, for example via Friedel-Crafts acylation or Vilsmeier-Haack formylation.

As already mentioned above, the difference between the method according to the invention and the methods of the prior art is that, inter alia, steps (A) and (B) (corresponding to steps 1 and 2 in exemplary FIG. 1) are carried out in a one-pot reaction. Thus, isolation of the intermediate from step (A) is no longer necessary, saving material, time, and energy.

In a preferred embodiment of the invention, the etherification (step A) is carried out using one or more compounds of the formula (II)

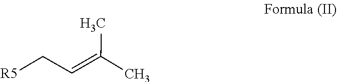

where R5 is chlorine, iodine or bromine. In a more preferred embodiment of the invention, R5 is chlorine. In other words, in a further preferred embodiment, the etherification is carried out with 1-chloro-3-methylbut-2-ene.

The phenol derivative is preferably selected from one or more compounds of the formula (III)

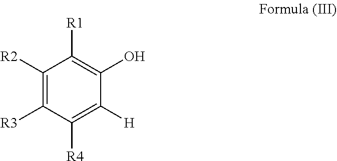

wherein for the radicals of the compound of the formula (III), it applies in each case that R1, R2, R3 and R4 are each independently hydrogen, methyl, ethyl, propyl, isopropyl, butyl, sec-butyl, tert-butyl, isobutyl, acetyl, formyl or cyano. In a further preferred embodiment of the invention, the aromatic alcohol is tert-butylphenol.

The etherification is preferably conducted at a reaction temperature of 90° C. to 130° C. In the most preferred embodiment of the invention, the etherification is conducted at 110° C.

Further preferred is the use of a base in the etherification. This base is preferably selected from the group consisting of potassium carbonate, sodium acetate, potassium acetate, potassium hydrogencarbonate, sodium carbonate or lithium carbonate. Particular preference is given to using potassium carbonate as base.

In a preferred embodiment of the invention, the Claisen rearrangement and subsequent cyclization of the intermediate from step (A), i.e. the allyl ether derivative, is carried out at 170° C. to 220° C. More preferably, the Claisen rearrangement and subsequent cyclization of the allyl ether derivative from step (A) is carried out at 190° C.

Preferably, the intermediate from step (B) is distilled prior to acetylation or formylation. Conventional methods are considered here, which are well known to those skilled in the art. Exemplary remarks in this regard can be found, inter alia, in the example section of this application.

In a preferred embodiment of the invention, a solvent is used in the etherification. The solvent is preferably selected from the group consisting of tetrahydrofuran, dimethylformamide, methylene chloride, 1,4-dioxane, N-methylpyrrolidone, acetonitrile, cyclohexanone and mixtures thereof. More preferably, N-methylpyrrolidone is used as solvent.

The compounds of the formula (I) are exceptionally suitable for use as fragrances and/or aromas with a musky scent note. The compounds of the formula (I) can be used alone or else together with other fragrances and/or aromas for the preparation of, for example, (perfumed) articles, e.g. perfume extracts, eau de parfums, eau de toilettes, aftershaves, eau de colognes, pre-shave products, splash colognes and scented refreshing wipes, and also the perfuming of acidic, alkaline and neutral cleaning agents such as floor cleaners, window cleaners, dishwashing detergents, bathroom and sanitary cleaners, scouring milk, solid and liquid toilet cleaners, pulverulent and foamed carpet cleaners, textile fresheners, ironing aids, liquid detergents, pulverulent detergents, laundry pretreatment agents such as bleach, softeners and stain removers, fabric softeners, laundry soaps, laundry tablets, disinfectants, surface disinfectants and air fresheners in liquid form, gel-like form or in a form applied to a solid carrier, aerosol sprays, waxes and polishes such as furniture polishes, floor waxes, shoe polishes and also personal care products such as solid and liquid soaps, shower gels, shampoos, shaving soaps, shaving foams, bath oils, cosmetic emulsions of the oil-in-water, water-in-oil and water-in-oil-in-water type such as skin creams and lotions, face creams and lotions, sunscreen creams and lotions, after-sun creams and lotions, hand creams and lotions, foot creams and lotions, depilatory creams and lotions, after-shave creams and lotions, tanning creams and lotions, hair care products such as hair sprays, hair gels, setting hair lotions, hair conditioners, permanent and semi-permanent hair colorants, hair shaping agents such as cold waves and hair straighteners, hair tonics, hair creams and lotions, deodorants and antiperspirants such as underarm sprays, roll-ons, deodorant sticks, deodorant creams, decorative cosmetics products such as eye shadow, nail varnish, make-up, lipstick, mascara, and also candles, lamp oils, incense sticks, insecticides, repellents and propellants.

In the following, the invention is further characterized using the working examples.

### EXAMPLES

### Example 1: Synthesis of Allyl Ether Derivatives

### 1-(7-tert-butyl-2,3,3-trimethyl-2H-benzofuran-5-yl)ethanone (Identified as 1 in FIG. 1)

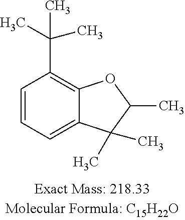 [SMILES: CC1Oc2c(C(C)(C)C)cccc2C1(C)C]

N-Methylpyrrolidone (81.6 g, 0.816 mol), potassium carbonate (27.4 g, 0.193 mol), tert-butylphenol (25.0 g, 0.165 mol) are initially charged at room temperature with vigorous stirring. The reaction mixture is heated to a bottom temperature of 110° C. and then 1-chloro-3-methylbut-2-ene (29.3 g, 0.249 mol) is metered in over a period of 8 hours. This is followed by a post-reaction time of 2 hours at the same bottom temperature.

### Example 2: Synthesis of Dihydrobenzofurans

### 7-tert-butyl-2,3,3-trimethyl-2H-benzofuran (Identified as 2 in FIG. 1)

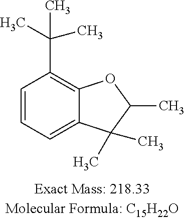 [SMILES: CC1Oc2c(C(C)(C)C)cccc2C1(C)C]

N-Methylpyrrolidone (52.5 g, 0.087 mol) and acetic acid (5.2 g, 0.087 mol) are added directly (one-pot reaction) to the reaction solution from Example 1. The mixture is then heated to a bottom temperature of 190° C. and the low boilers are distilled off. This is followed by a post-reaction time of 8 hours at the same bottom temperature. 100 ml of MTBE are then added to the reaction solution at room temperature, the organic phase is washed twice with 100 ml of 10% NaCl and concentrated on the rotary evaporator. The crude product (50.0 g) is distilled in a Kugelrohr distillation system.

Yield: 40.0 g of 7-tert-butyl-2,3,3-trimethyl-2H-benzofuran as a colorless oil (97.2% of theory) b.p.: 110° C./0.8 mbar

GC evaluation (20 m DB-1, internal diameter 0.2 μm/60-9-240° C. cold feed system)

### Example 3: Synthesis of Compound 1

### 1-(7-tert-butyl-2,3,3-trimethyl-2H-benzofuran-5-yl)ethanone (Compound 1 in FIG. 1)

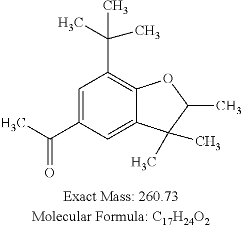 [SMILES: CC(=O)c1cc(C(C)(C)C)c2c(c1)C(C)(C)C(C)O2]

Acetyl chloride (13.3 g, 0.170 mol) is added dropwise, with cooling, to a suspension of dichloromethane (85 g) and aluminum chloride (25.6 g, 0.192 mol). The distillate from Example 2 (40.0 g, 0.160 mol) is then metered in up to a maximum reaction temperature of 20° C. and the mixture is stirred overnight at the same temperature. 100 g of dichloromethane and water are then added to the reaction solution while cooling with ice. The organic phase is neutralized and concentrated. The crude product (39.2 g) is distilled in a Kugelrohr distillation system.

Yield: 36.8 g of 1-(7-tert-butyl-2,3,3-trimethyl-2H-benzofuran-5-yl)ethanone as a colorless oil (73.2% of theory, based on use formula 2) b.p.: 140° C./0.8 mbar

GC evaluation (20 m DB-1, internal diameter 0.2 μm/60-9-240° C. cold feed system)

1. A method for preparing a compound of the formula (I) 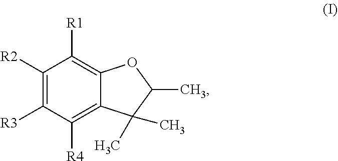
wherein R1, R2, R3 and R4 are each independently hydrogen, methyl, ethyl, propyl, isopropyl, butyl, sec-butyl, tert-butyl, isobutyl, acetyl, formyl or cyano,
comprising the following steps:
(A) etherification of a phenol derivative to obtain an allyl ether derivative;
(B) Claisen rearrangement and subsequent cyclization of the ether from step (A); and
(C) acetylation or formylation of the intermediate from step (B),
wherein the steps (A) and (B) are carried out in a one-pot reaction.

2. The method as claimed in claim 1, wherein
R1 is methyl, ethyl, propyl, isopropyl, butyl, sec-butyl, tert-butyl or isobutyl, preferably methyl, ethyl, isopropyl or tert-butyl, and/or
R2 is hydrogen, methyl, formyl or acetyl, preferably hydrogen, formyl or acetyl, and/or
R3 is hydrogen, methyl, formyl or acetyl, preferably hydrogen, formyl or acetyl, and/or
R4 is hydrogen, methyl, formyl or acetyl, preferably hydrogen, formyl or acetyl.

3. The method as claimed in claim 1, wherein the compound of formula (I) is selected from the group consisting of the following compounds 1 to 3:

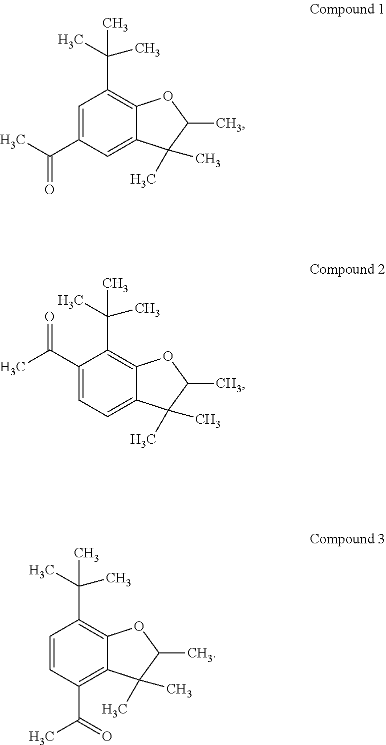 [SMILES: CC(=O)c1cc(C(C)(C)C)c2c(c1)C(C)(C)C(C)O2 ; CC(=O)c1ccc2c(c1C(C)(C)C)OC(C)C2(C)C ; CC(=O)c1ccc(C(C)(C)C)c2c1C(C)(C)C(C)O2]

4. The method as claimed in claim 1, wherein the etherification is carried out with one or more compounds of the formula (II) 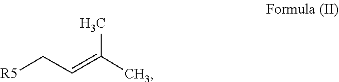
wherein R5 is chlorine, iodine or bromine.

5. The method as claimed in claim 4, wherein R5 is chlorine.

6. The method as claimed in claim 1, wherein the phenol derivative is selected from one or more compounds of the formula (III) 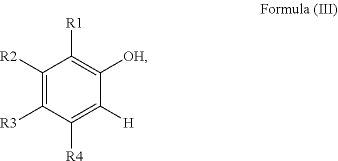
wherein R1, R2, R3 and R4 are each independently hydrogen, methyl, ethyl, propyl, isopropyl, butyl, sec-butyl, tert-butyl, isobutyl, acetyl, formyl or cyano.

7. The method as claimed in claim 1, wherein the phenol derivative is tert-butylphenol.

8. The method as claimed in claim 1, wherein the reaction temperature in the etherification is from 90° C. to 130° C.

9. The method as claimed in claim 1, wherein a base is used in the etherification.

10. The method as claimed in claim 10, wherein the base is selected from the group consisting of potassium carbonate, sodium acetate, potassium acetate, potassium hydrogencarbonate, sodium carbonate, lithium carbonate, and mixtures thereof.

11. The method as claimed in claim 1, wherein the Claisen rearrangement and subsequent cyclization of the ether from step (A) is carried out at 170° C. to 220° C.

12. The method as claimed in claim 11, wherein the Claisen rearrangement and subsequent cyclization of the ether from step (A) is carried out at 190° C.

13. The method as claimed in claim 1, wherein the intermediate from step (B) is distilled prior to acetylation or formylation.

14. The method as claimed in claim 1, wherein a solvent is used in the etherification is selected from the group consisting of tetrahydrofuran, dimethylformamide, methylene chloride, 1,4-dioxane, N-methylpyrrolidone, acetonitrile, cyclohexanone, and mixtures thereof.

15. The method as claimed in claim 14, wherein the solvent is N-methylpyrrolidone.
